(19) United States
(12) Patent Application Publication
(10) Pub. No.: US 2025/0235290 A1
(43) Pub. Date: Jul. 24, 2025

(54) BIOPSY DEVICE WITH END DEPLOYMENT FOR MARKER DELIVERY

(71) Applicant: Devicor Medical Products, Inc., Cincinnati, OH (US)

(72) Inventors: Jack A. Randall, Cincinnati, OH (US); Andrew P. Nock, Dayton, OH (US); Garrett A. Householder, Cincinnati, OH (US)

(21) Appl. No.: 19/171,629

(22) Filed: Apr. 7, 2025

Related U.S. Application Data
(63) Continuation of application No. PCT/US2023/035033, filed on Oct. 12, 2023.
(60) Provisional application No. 63/418,297, filed on Oct. 21, 2022.

(51) Int. Cl. A61B 90/00; A61B 10/02

(52) U.S. Cl. CPC A61B 90/39; A61B 10/0275; A61B 2090/3925; A61B 2090/3966; A61B 2090/3987; A61B 2090/3995

(57) ABSTRACT

A system for marking a biopsy site includes a biopsy device and a biopsy site marker. The biopsy device includes a body and a needle. The needle extends distally from the body and includes a tissue piercing tip. The needle defines an axial lumen and a lateral lumen. The axial lumen is configured to receive a cutter therein. The tissue piercing tip defines a marking lumen in communication with the lateral lumen. The biopsy site marker includes a non-absorbable marker element and a bioabsorbable carrier. The marker element is at least partially disposed within the carrier. The biopsy site marker is configured for receipt within the marking lumen of the needle.

### PRIORITY

This application claims priority to U.S. Provisional Application Ser. No. 63/418,297, entitled “Biopsy Device with End Deployment for Marker Delivery,” filed on Oct. 21, 2022, the disclosure of which is incorporated by reference herein.

### BACKGROUND

A number of patients will have breast biopsies because of irregular mammograms and palpable abnormalities. Biopsies can include surgical excisional biopsies and stereotactic and ultrasound guided needle breast biopsies. In the case of image directed biopsy, the radiologist or other physician may take a small sample of the irregular tissue for laboratory analysis. If the biopsy proves to be malignant, additional surgery (e.g., a lumpectomy or a mastectomy) may be required. In the case of needle-based biopsies, the patient may return to the radiologist a day or more later, and the biopsy site (the site of the lesion) may need to be relocated in preparation for the surgery. An imaging system, such as ultrasound, magnetic resonance imaging (MRI) or x-ray may be used to locate the biopsy site. In order to assist the relocation of the biopsy site, a marker may be placed at the time of the biopsy.

The use of markers used after breast biopsies to mark the location where the biopsied tissue was removed is described in the following US Patents: US Pat. No. 6,083,524, entitled “Polymerizable biodegradable polymers including carbonate or dioxanone linkages,” issued Jul. 4, 2000; US Pat. No. 6,162,241, entitled “Hemostatic tissue sealants,” issued Dec. 4, 2000; US Pat. No. 6,270,464, entitled “Biopsy localization method and device,” issued Aug. 7, 2001; US Pat. No. 6,356,782, entitled “Subcutaneous cavity marking device and method,” issued Mar. 12, 2002; US Pat. No. 6,605,294, entitled “Methods of using in situ hydration of hydrogel articles for sealing or augmentation of tissue or vessels,” issued Aug. 12, 2003; US Pat. No. 8,600,481, entitled “Subcutaneous cavity marking device,” issued Dec. 3, 2013 and US Pat. No. 8,939,910, entitled “Method for enhancing ultrasound visibility of hyperechoic materials”, issued Jan. 27, 2015. All of these US Patents are incorporated by reference in their entirety.

Examples of breast biopsy devices used in connection with marker deployment are described in US Pub. No. 2009/0216151, entitled “Biopsy Probe with Hypodermic Lumen,” published Aug. 27, 2009; and US Pub. No. 2021/0282754, entitled “Biopsy System with End Deploy Needle,” published Sep. 16, 2021, the disclosures of which are incorporated by reference herein in their entirety.

Placement of a marker at the biopsy site may be performed through the biopsy needle itself or other elements associated with the biopsy needle such as an introducer cannula, a targeting sheath, obturator, and/or etc. In some circumstances, the particular configuration of the device used for placement of the marker may present challenges with the placing the marker at the biopsy site reliably and accurately. For instance, when the biopsy needle is used for placement of the marker, the marker may be driven through a lateral aperture in the biopsy needle. This form of deployment of the marker may be desirable because the lateral aperture of the biopsy needle may also be used to collect biopsy samples, so its position may directly correspond to the biopsy site. However, there may be challenges associated with directing the marker axially through the biopsy needle and then laterally out of the lateral aperture. Accordingly, it may be desirable to use alternative features to deploy a marker to increase reliably and accuracy of placement of the marker.

While several systems and methods have been made and used for obtaining a biopsy sample and marking a biopsy site, it is believed that no one prior to the inventors has made or used the invention described in the appended claims.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements. In the drawings some components or portions of components are shown in phantom as depicted by broken lines.

FIG. 1 depicts a front elevational view of an example of a biopsy site marker;

FIG. 2 depicts a perspective view of an example of a biopsy probe for use with the marker of FIG. 1;

FIG. 3 depicts an exploded perspective view of a tissue sample holder of the biopsy probe of FIG. 2;

FIG. 4 depicts a detailed perspective view of a needle of the biopsy probe of FIG. 2;

FIG. 5 depicts a side cross-sectional view of the needle of FIG. 4, the cross-section taken along line 5-5 of FIG. 4;

FIG. 6A depicts another side cross-sectional view of the needle of FIG. 4, the needle having the marker of FIG. 1 loaded therein;

FIG. 6B depicts yet another side cross-sectional view of the needle of FIG. 4, the needle being used to deploy the marker of FIG. 1 at a biopsy site;

FIG. 7 depicts a detailed perspective view of an example of an alternative needle for use with the biopsy probe of FIG. 2;

FIG. 8 depicts another detailed perspective view of the needle of FIG. 7;

FIG. 9 depicts a side cross-sectional view of the needle of FIG. 7, the cross-section taken along line 9-9 of FIG. 7;

FIG. 10A depicts another side cross-sectional view of the needle of FIG. 7, the needle being used in combination with the marker of FIG. 1;

FIG. 10B depicts yet another side cross-sectional view of the needle of FIG. 7, the marker of FIG. 1 being deployed from the needle;

FIG. 11 depicts a detailed perspective view of an example of another alternative needle for use with the biopsy probe of FIG. 2;

FIG. 12 depicts a side cross-sectional view of the needle of FIG. 11, the cross-section being taken along line 12-12 of FIG. 11;

FIG. 13A depicts another side cross-sectional view of the needle of FIG. 11, the needle being used in combination with the marker of FIG. 1; and

FIG. 13B depicts another side cross-sectional view of the needle of FIG. 11, the marker of FIG. 1 being deployed from the needle.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

It may be beneficial to be able to mark the location or margins of a lesion, whether temporarily or permanently, prior to or immediately after removing or sampling it. Marking prior to removal may help to ensure that the entire lesion is excised, if desired. Alternatively, if the lesion were inadvertently removed in its entirety, marking the biopsy site immediately after the procedure would enable reestablishment of its location for future identification.

Once a marker is positioned at a biopsy site, it may be desirable for the marker to remain visible under ultrasound. It may also be desirable to make the marker readily identifiable relative to other structural features of a patient. For instance, it may be desirable for the marker to be distinguishable under ultrasound visualization from microcalcifications to avoid inadvertently characterizing the marker as a microcalcification during subsequent ultrasonic examinations. Generally, microcalcifications are used in the field to identify suspicious lesions or masses. Thus, it is generally desirable for the ultrasound view to be distinguishable as a marker and not inadvertently identified as a new mass.

### I. Exemplary Marker

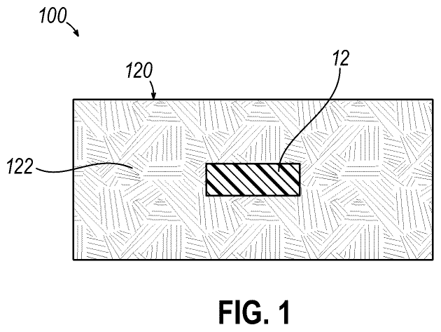

Aspects presented herein relate to devices and procedures for manufacturing a marker for percutaneously marking a biopsy cavity. For instance, as seen in FIG. 1, a marker (100) may be initially placed in the biopsy cavity to facilitate relocation of the biopsy site. Marker (100) may include a carrier (120) and a marker element (12). Carrier (120) generally includes a bioabsorbable marker material (122). Thus, carrier (120) is generally configured for absorption into a patient after placement of marker (100) within the biopsy cavity. In some versions, carrier (120) can include a plurality of microbubbles to enhance visualization of carrier (120) under ultrasound. As will be described in greater detail below, marker material (122) is generally bioabsorbable such that marker material (122) may be generally absorbed into the patient's tissue over time. In the present example, marker material (122) comprises a hydrogel that is initially in a dehydrated state. Although a hydrogel is used in the present example, it should be understood that in other examples marker material (122) may comprise other known bioabsorbable materials such as collagen, certain synthetic materials, or combinations of hydrogel with other materials.

In the present version, marker (100) further includes a marker element (12) that is generally not bioabsorbable. Marker element (12) may comprise a radiopaque or echogenic marker embedded within the bioabsorbable marker material (122) of carrier (120). For instance, marker element (12) may include metal, hard plastic, or other radiopaque or hyperechoic materials known to those of ordinary skill in the art in view of the teachings herein. In other versions, marker (100) may be formed without a marker element (12). In still other versions, marker (100) may be formed with only marker element (12) such that carrier (120) is omitted and marker element (12) is in a “bare” form. In other words, in some examples, marker (100) is formed of only carrier (120) as a bare clip.

Marker material (122) is generally expandable once disposed within a patient at a biopsy site. In particular, the initially dehydrated marker material (122) may absorb fluid from the surrounding tissue into which it is inserted. In response to this absorption of fluid, marker material (122) may swell, thereby permitting carrier (120) to fill a cavity formed at a biopsy site by removal of tissue samples during a biopsy procedure. Biodegradable materials may be particularly suitable in applications where it is desired that natural tissue growth be permitted to completely or partially replace the implanted material over time. Accordingly, biocompatibility is ensured, and the natural mechanical parameters of the tissue are substantially restored to those of the pre-damaged condition.

Marker (100) may be inserted into the body either surgically via an opening in the body cavity, or through a minimally invasive procedure using such devices as a catheter, introducer or similar type insertion device. Marker (100) may be delivered immediately after removal of the tissue specimen using the same device used to remove the tissue specimen itself. Follow-up noninvasive detection techniques, such as x-ray mammography or ultrasound may then be used by the physician to identify, locate, and monitor the biopsy cavity site over a period of time via marker (100).

Marker (100) of the present version is large enough to be readily visible to a clinician under x-ray or ultrasonic viewing, for example; yet small enough to be able to be percutaneously deployed into the biopsy cavity and to not cause any difficulties with the patient. Although examples are described in connection with treatment and diagnosis of breast tissue, aspects presented herein may be used for markers in any internal, tissue, e.g., in breast tissue, lung tissue, prostate tissue, lymph gland tissue, etc.

The hydration of the marker material (122) of carrier (120) by the natural moisture of the tissue surrounding it causes expansion of the polymer and thus minimizes the risk of migration. The growing hydrogel-based marker material (122) centers marker (100) in the biopsy cavity as it grows. As the hydrogel expands, naturally present moisture from the surrounding tissue, the hydration enables increasing sound through transmission, appears more and more hypoechoic and is easy to visualize on follow-up ultrasound studies.

The hydrated hydrogel marker material (122) of carrier (120) may also be used to frame permanent marker (12). The hypoechoic nature of the hydrated marker material (122) enables ultrasound visibility of the permanent marker (12) within the hydrogel hydrated marker material (122) because the permanent marker (12) is outlined as a specular reflector within a hypoechoic hydrated marker having a water-like nonreflective substrate.

### II. Example Biopsy Probe With End Deployment Features for Marker

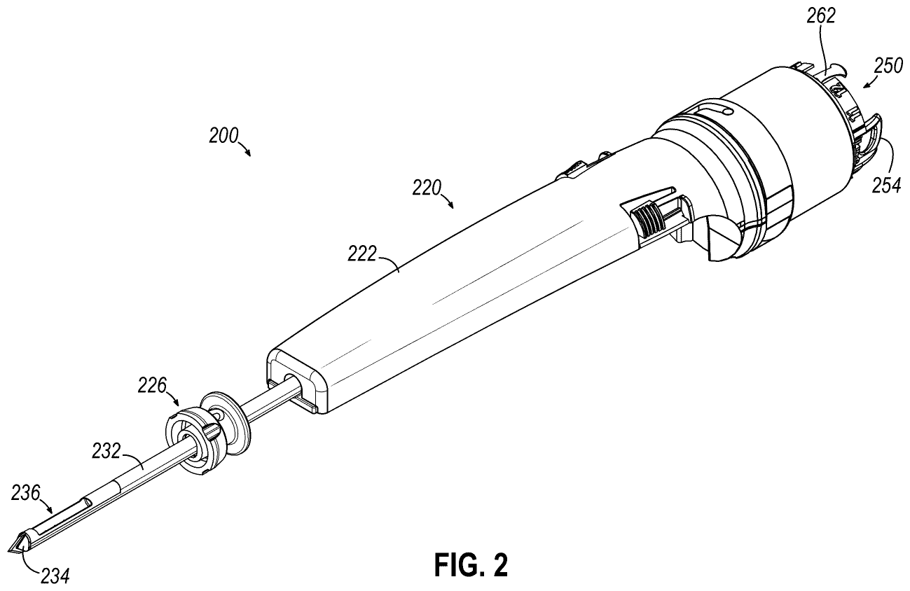

FIG. 2 shows an example of a biopsy probe (220) for use with marker (100). Although not shown, it should be understood that biopsy probe (220) may be used in connection with a broader biopsy device. Such a biopsy device may include a holster (not shown) in addition to probe (220). In such a biopsy device, probe (220) may be separable from the holster. By way of example only, probe (220) may be provided as a disposable component, while the holster may be provided as a reusable component. Use of the term “holster” herein should not be read as requiring any portion of probe (220) to be inserted into any portion of the holster. Indeed, in some versions of a biopsy device, probe (220) may simply sit on the holster. In some other versions, a portion of the holster may be inserted into probe (220). Furthermore, in some versions, probe (220) and the holster may be of unitary or integral construction, such that the two components cannot be separated or are not identifiable as different components. Still other suitable structural and functional relationships between probe (220) and the holster will be apparent to those of ordinary skill in the art in view of the teachings herein.

Although not shown, it should be understood that a suitable holster may be configured to drive various functions of probe (220). To faciliate such functions, suitable holsters may thus include a needle rotation mechanism (not shown), a needle firing mechanism (not shown), a cutter drive mechanism (not shown), and a tissue holder rotation mechanism (not shown). The needle rotation mechanism may be operable to rotate a needle (226) about its longitudinal axis. The needle firing mechanism may be operable to fire needle (226) into tissue. The cutter drive mechanism may be operable to cause a cutter (246) to rotate and translate; while the tissue holder rotation mechanism may be operable to cause at least a portion of a tissue sample holder (250) to rotate. Suitable components and structures that may be used to provide any of these mechanisms, as well as other optional features of suitable holsters, are disclosed in U.S. Non-Provisional patent application Ser. No. 11/942,764, filed Nov. 20, 2007, and entitled “Vacuum Timing Algorithm for Biopsy Device,” the disclosure of which is incorporated by reference herein. Of course, any other suitable components, structures, or configurations may be used. Alternatively, any such mechanisms may simply be omitted altogether.

Probe (220) includes a body (222) and a needle (210) extending distally from body (222). Probe (220) may further include a tissue sample holder (250). Tissue sample holder (250) may be removably secured to body (222), though tissue sample holder (250) may alternatively be secured to some other component. Although not shown, it should be understood that one or more may be coupled with probe (220) for communication of fluids (e.g., vacuum, saline, atmospheric air, pressurized air, etc.) to needle (226) and/or tissue sample holder (250).

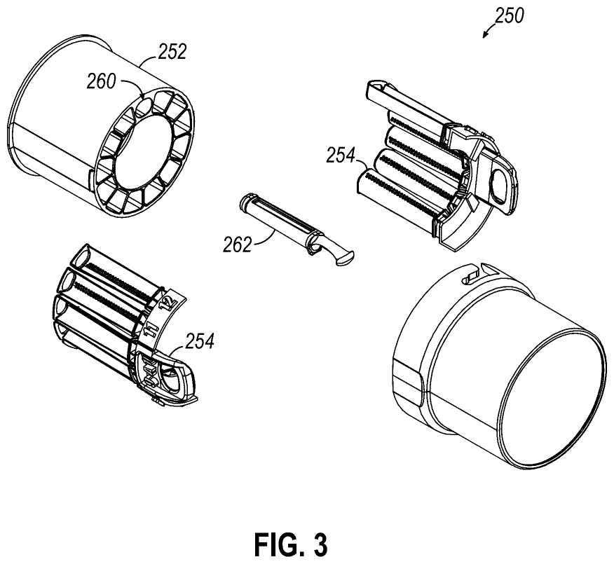

FIG. 3 shows tissue sample holder (250) is shown in greater detail. Tissue sample holder (250) is configured to receive one or more tissue samples severed by a hollow tubular cutter (246), as will be described in greater detail below. Such tissue samples may be transported through a hollow interior of cutter (246) and deposited within a portion of tissue sample holder (250). It should be understood that tissue sample holder (250) may take on a variety of forms and is not limited by the particular configuration shown and described herein. In the present version, tissue sample holder (250) includes a rotatable member (252) that is configured to receive one or more sample trays (254). Each sample tray (254) may include a plurality of strips (255). Each strip (255) may be received in a corresponding sample chamber (253) defined by rotatable member (252). Each strip (255) is thus configured to receive one or more tissue samples as rotatable member (252) is rotated to successively fill each strip with one or more severed tissue samples.

Rotatable member (252) further includes an access port (260) configured to removably receive an access plug (262). As will be described in greater detail below, access port (260) is configured to selectively provide access to the hollow interior of cutter (246) depending on the rotational position of rotatable member (252). Access port (260) in the present version is disposed between two sample trays (254). In this position, rotatable member (252) may be rotated to align access port (260) with a proximal end of cutter (246) rather than a sample chambers (253) and a corresponding strip (255). Once aligned, access port (260) may be in communication with the hollow interior of cutter (246). In other versions, access port (260) may be positioned separately from sample trays (254) to promote access to the hollow interior of cutter (246) while still permitting sample collection using sample trays (254).

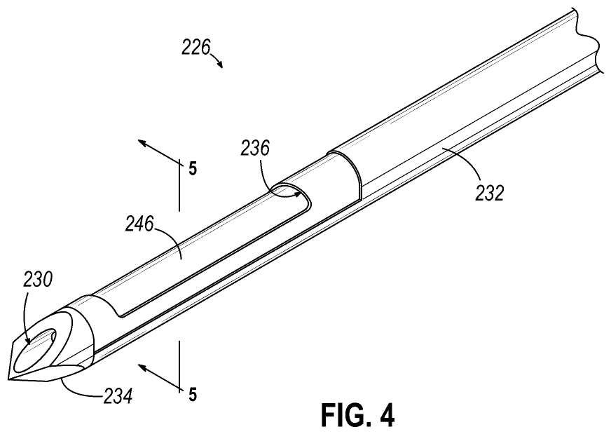

As best seen in FIG. 4, needle (226) includes an outer cannula (232) having a tissue piercing tip (234) and a transverse tissue receiving aperture (236) (also referred to as a lateral aperture). The interior of outer cannula (232) of the present version defines a cannula lumen (238) (also referred to as a cutter lumen or axial) and a vacuum lumen (240) (also referred to as a lower lumen or lateral lumen), with a wall (242) separating cannula lumen (238) from vacuum lumen (240). A plurality of fluid openings (244) may be formed in wall (242) to provide fluid communication between cannula lumen (238) and vacuum lumen (240). Of course, as with other components described herein, such openings are merely optional.

A hollow tubular cutter (246) may be slidably disposed within cannula lumen (238) of needle (226). Cutter (246) is generally configured to rotate and translate relative to needle (226) to sever one or more tissue samples from tissue prolapsed into aperture (236) of needle (226). Thus, cutter (246) may translate relative to aperture (236) to open and close aperture (236) either to sever tissue samples or to faciliate one or more other functions during a biopsy procedure. As described above, cutter (246) may be driven by one or more components of a holster. For instance, in some versions, a cutter drive (not shown) may be partially disposed within body (222). Such a cutter drive may then be driven by one or more gears extending from a portion of the holster.

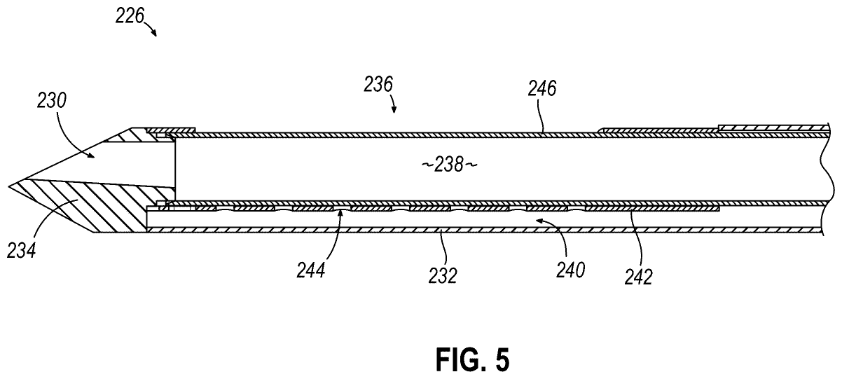

As best seen in FIGS. 4 and 5, needle (226) further includes a marking lumen (230) extending through tissue piercing tip (234). As will be described in greater detail below, marking lumen (230) is generally configured to permit deployment of marker (100) through tissue piercing tip (234). Marker lumen (230) is positioned within tissue piercing tip (234) to communicate with cannula lumen (238). Marker lumen (230) is further positioned within tissue piercing tip (234) to align with a generally planar surface of tissue piercing tip (234) rather than intersecting with one or more cutting edges. In this configuration, isolation of marker lumen (230) to a planar surface of tissue piercing tip (234) may be desirable to preserve the tissue piercing function of tissue piercing tip (234) while providing access to cannula lumen (238).

Although marking lumen (230) of the present version is configured to communicate with cannula lumen (238), it should be understood that marker lumen (230) may be in communication with vacuum lumen (240) in other versions. In still other versions, marking lumen (230) may be configured to communicate with an entirely separate lumen extending through needle (226). For instance, in such versions, needle (226) may include an additional tubular structure adjacent to portions of wall (242) and/or outer cannula (232). Such a tubular structure may be configured to faciliate the transport of marker (100) through needle (226) to making lumen (230).

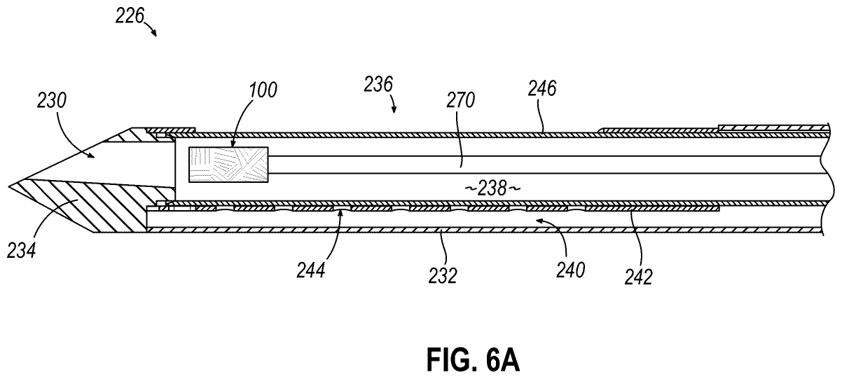

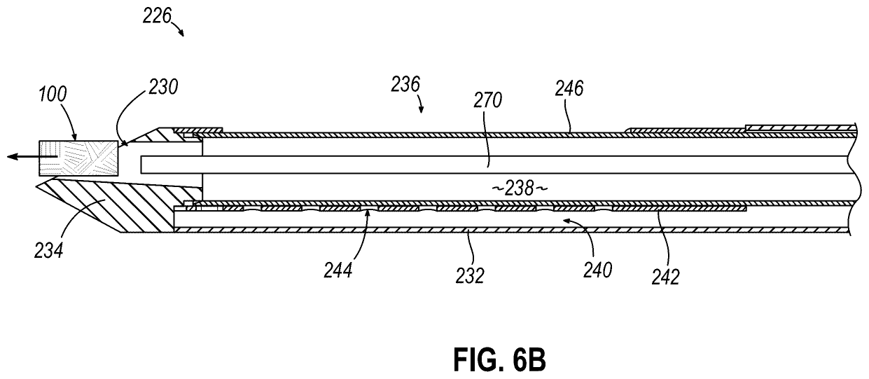

FIGS. 6A and 6B show an example of a use of needle (226) to deploy marker (100) at a biopsy site. Although not shown, it should be understood that prior to deployment of marker (100), biopsy device (200) may be used to prepare the biopsy site. For instance, needle (226) may be used to collect one or more tissue samples through aperture (236) using cutter (246). Once a desired number of tissue samples are collected, needle (226) may remain in position at the biopsy site for the purpose of marking. Because needle (226) remains in position at the biopsy site, marking using needle (226) may substantially correspond to the area of collection of tissue samples.

Prior to marking, tissue sample holder (250) may also be prepared to receive marker (100). For instance, rotatable member (252) may be rotated to align access port (260) with cutter (246). At this stage, cutter (246) may also be advanced distally to close aperture (236) of needle (226) as shown in FIG. 6A. Access plug (262) may then be removed to provide a continuous path from the proximal end of tissue sample holder (250), through cutter (246), and to marking lumen (230).

After preparation of biopsy device (200) for marking, marker (100) may be introduced into needle (226) through access port (260) of tissue sample holder (250). Marker (100) may then be moved distally through cutter (246) toward marking lumen (230) as shown in FIG. 6A. In the present use, a push rod (270) may be used to advance maker (100) distally though needle (226). Push rod (270) may be configured as an elongate rod with sufficient rigidity to push marker (100), yet with sufficient flexiblity to be manipulated through one or more portions of biopsy device (200). Although not shown, it should be understood that in some versions, push rod (270) may take on a variety of alternative forms. For instance, in some versions, a portion of push rod (270) may include a sled feature or marker coupling feature configured to hold marker (100) to push rod (270) until deployment. Such sled features may include cams, resilient portions, and/or living hinges to faciliate engagement with internal structures of needle (226) and release of marker (100) from push rod (270).

Although marker (100) is manipulated in the present use by a mechanical mechanism such as push rod (270), it should be understood that in other versions various alternative manipulation mechanisms and mediums may be used. For instance, in some versions, a pressurized fluid may be used in lieu of push rod (270). In other versions, pneumatics may be used to manipulate marker (100) either in combination with one or more mechanical mechanisms or with pneumatics alone. Of course, additional alternative mechanisms for manipulation of maker (100) will be apparent to those of ordinary skill in the art in view of the teachings herein.

Regardless of the particular mechanism used for manipulation of marker (100), marker (100) may subsequently be advanced through marking lumen (230) as shown in FIG. 6B. As can be seen, marker (100) may be advanced distally out of tissue piercing tip (234) and into the biopsy site. In the present version, push rod (270) may be used to advance marker (100) through marking lumen (230). In some versions, push rod (270) may be partially extended out of tissue piercing tip (234) to ensure complete deployment of marker (100). Subsequently, push rod (270) may be retracted back into marking lumen (230). After retraction of push rod (270) one or more subsequent markers similar to marker (100) may be optionally deployed using push rod (270)

After completion of marking, needle (226) may be withdrawn from the tissue, while marker (100) may remain in position at the biopsy site. After the biopsy procedure, marker (100) may be used to identify the biopsy site in subsequent follow-up procedures. Identification of marker (100) may be performed using a variety of imaging modalities such as ultrasound, x-ray, MRI, and/or etc.

### III. Examples of Alternative Needles With End Deployment Features

As described above, it may be desirable to deploy marker (100) through a structure of a needle similar to tissue piercing tip (234) rather than through a structure similar to aperture (236). Such a method of deployment may be desirable to provide the accuracy of marker placement generally associated with deployment through a needle while also providing improved reliability of deployment. Although some examples of a needle with marker deployment through a structure similar to tissue piercing tip (234) are described above, other needle configurations may provide similar benefits, while also being more complementary with other functions of the needle. While some examples of suitable needles are described below, it should be understood that various needle configurations may be implemented without departing from the features and concepts described herein.

### A. Example of Alternative Needle With Lateral Marking Lumen

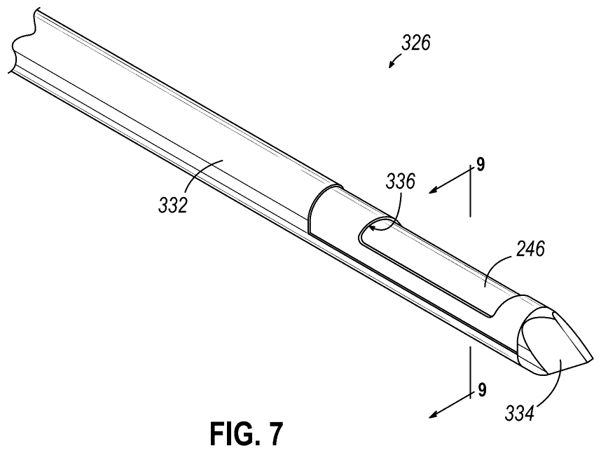

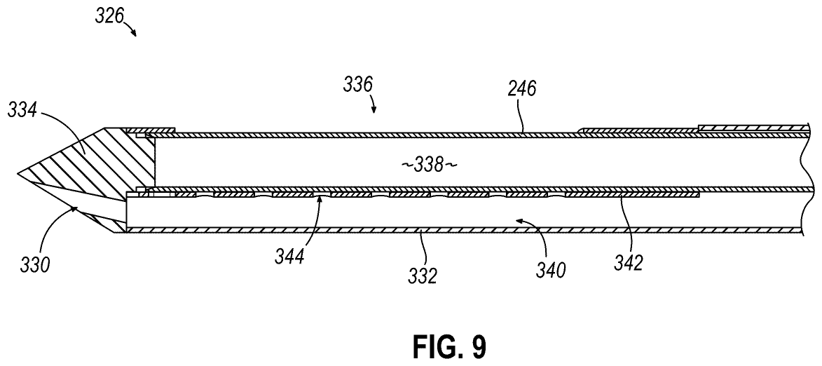

FIGS. 7 through 9 show an example of an alternative needle (326) that may be incorporated into biopsy device (200) described above in lieu of needle (226). Needle (326) of the present version is substantially similar to needle (226) described above, except where otherwise described herein. For instance, like with needle (226) described above, needle (326) of the present version includes an outer cannula (332) having a tissue piercing tip (334) and a transverse tissue receiving aperture (336) (also referred to as a lateral aperture). As with outer cannula (232) described above, the interior of outer cannula (332) of the present version defines a cannula lumen (338) (also referred to as a cutter lumen or axial lumen) and a vacuum lumen (340) (also referred to as a lower lumen or lateral lumen), with a wall (342) separating cannula lumen (338) from vacuum lumen (340). A plurality of fluid openings (344) may be formed in wall (342) to provide fluid communication between cannula lumen (338) and vacuum lumen (340). Of course, as with other components described herein, such openings are merely optional.

Similar to needle (226) described above, needle (326) of the present version includes a marking lumen (330) extending through a portion of tissue piercing tip (334) and in communication with the interior of needle (326). As with marking lumen (230) described above, marking lumen (330) of the present version is generally configured to receive marker (100) so that marker (100) may be deployed through tissue piercing tip (334). Also like with marking lumen (230) described above, marking lumen (330) may be positioned relative to tissue piercing tip (334) so as to intersect with only a planar surface and not any cutting edge. As described above, such a configuration may be desirable to promote the tissue piercing function of tissue piercing tip (334), while also permitting deployment of marker (100) through tissue piercing tip (334).

Unlike marking lumen (230) described above, marking lumen (330) of the present version is oriented on an opposite side of tissue piercing tip (334) relative to marking lumen (230) described above. In other words, marking lumen (330) is positioned to extend through a lower portion of tissue piercing tip (334) rather than an upper portion like marker lumen (230) described above. In this position, marking lumen (330) is positioned to communicate with vacuum lumen (340) of needle (326) rather than cannula lumen (338). As will be described in greater detail below, this feature may be desirable to permit transport of marker (100) to marking lumen (330) through vacuum lumen (340) rather than cannula lumen (338) and cutter (246).

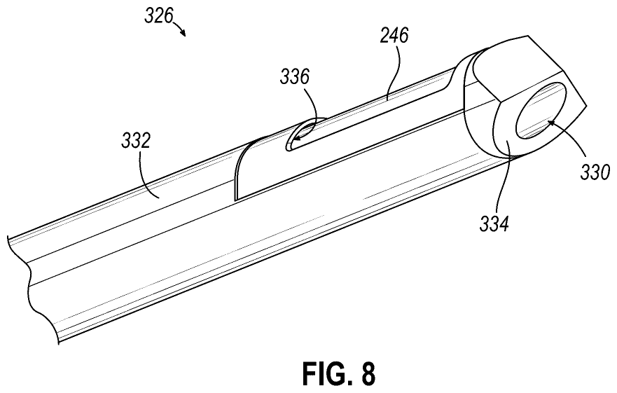

It should be understood that in versions where marking lumen (330) is repositioned, various elements of piercing tip (334) may also be repositioned. For instance, as best seen in FIG. 8, the position of marking lumen (330) proximate the lower portion of tissue piercing tip (334) may result in a planar surface of tissue piercing tip (334) being associated with the bottom portion of tissue piercing tip (334) rather than an upper portion of tissue piercing tip (334). Thus, in the present version, one or more planar surface of tissue piercing tip (334) may be rearranged relative to tissue piercing tip (234) described above to correspond to the lower portion of tissue piercing tip (334). Meanwhile, one or more edges of tissue piercing tip (334) may likewise be rearranged to accommodate the rearrangement of planar surfaces.

Although not shown, it should be understood that the proximal end of vacuum lumen (340) may be configured to communicate either directly with a feature of a marker delivery device (not shown) such as push rod (270), or indirectly through a portion of tissue sample holder (250) or a port proximate tissue sample holder (250). For instance, in some versions, a structure similar to push rod (270) may be incorporated into probe (220) or the holster. Such a structure may be directly in communication with vacuum lumen (340) and may be actuated for marking purposes. In such versions, marker (100) may be preloaded within a portion of biopsy device (200). Alternatively, marker (100) may be loaded manually through a port disposed within a portion of biopsy device (200). In other versions, tissue sample holder (250) may be configured to communicate with vacuum lumen (340) via access port (260) similarly to communication with cutter (246) but offset relative to the position described above with respect to cutter (246) communication. In yet other versions, vacuum lumen (340) may be coupled to a tube (not shown), which may extend proximally from a portion of probe (220). Such a tube may be configured to receive marker (100) and/or other components associated with marker (100) to faciliate transport of marker (100) through vacuum lumen (340) to marking lumen (230).

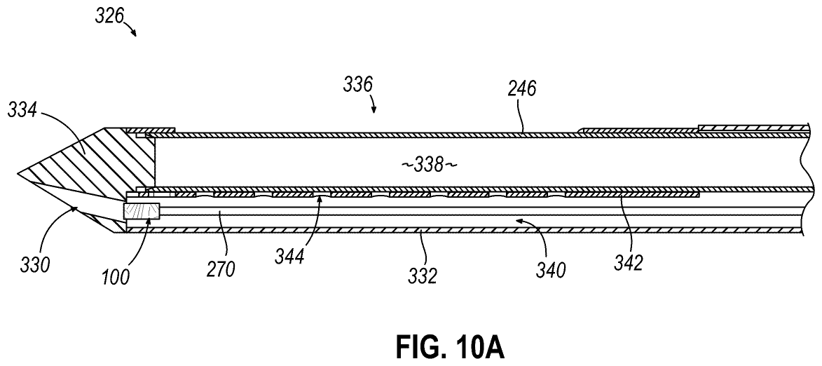

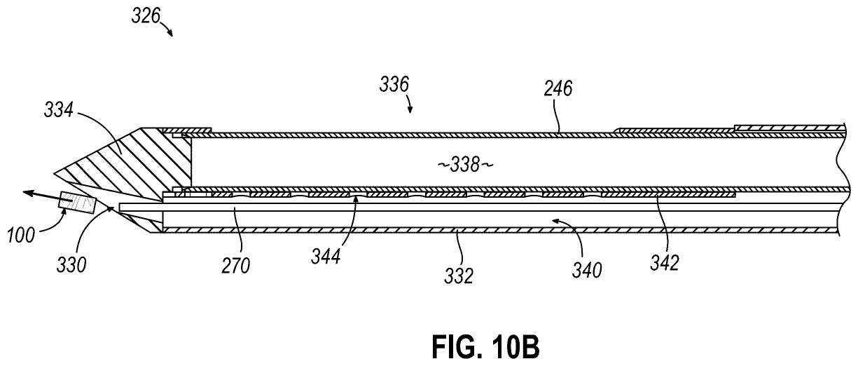

FIGS. 10A through 10B show an example of a use of needle (326) for deployment of marker (100) within tissue. The use with respect to needle (326) of the present version is substantially similar to the use described above with respect to needle (226). For instance, as described above, biopsy device (200) may initially be prepared for marking. Such initial preparations may include, for example, collecting one or more tissue samples, aligning tissue sample holder (250) to communicate with vacuum lumen (340), and/or inserting features associated with marker such as push rod (270) into biopsy device (200) to communicate marker (100) through vacuum lumen (340).

Once such initial preparations are completed, marker (100) may be advanced through vacuum lumen (340) as shown in FIG. 10A. As similarly described above with respect to needle (226), marker (100) may be advanced using push rod (270) as shown or with other suitable advancement modes such as pressurized fluids, pneumatics, and/or pneumatic mechanisms. Optionally, in some versions, marker (100) and/or push rod (270) may be preloaded as shown in FIG. 10A prior to performing a biopsy procedure. Such a preloaded configuration may be desirable in the present version to simplify the biopsy procedure. Additionally, because vacuum lumen (340) is not used to physically transport tissue samples, the presence of marker (100) and/or push rod (270) may have limited impact on a biopsy procedure, even during sample collection. Although not shown, it should be understood that in preloaded versions, marking lumen (330), vacuum lumen (340), or both may include features configured to maintain the position of marker (100) within needle (326) until deployment of marker (100) is desired. Suitable features configured to maintain the position of maker (100) may include, for example, detents, ridges, protrusions, resilient flaps, and/or etc.

Once marker (100) is advanced through vacuum lumen (340) to marking lumen (330), marker (100) may subsequently be advanced through marking lumen (330) as shown in FIG. 10B. As can be seen, marker (100) may be advanced distally out of tissue piercing tip (334) and into the biopsy site. In the present version, push rod (270) may be used to advance marker (100) through marking lumen (330). In some versions, push rod (270) may be partially extended out of tissue piercing tip (334) to ensure complete deployment of marker (100). Subsequently, push rod (270) may be retracted back into marking lumen (330). After retraction of push rod (270) one or more subsequent markers similar to marker (100) may be optionally deployed using push rod (270).

After completion of marking, needle (326) may be withdrawn from the tissue, while marker (100) may remain in position at the biopsy site. After the biopsy procedure, marker (100) may be used to identify the biopsy site in subsequent follow-up procedures. Identification of marker (100) may be performed using a variety of imaging modalities such as ultrasound, x-ray, MRI, and/or etc.

### B. Example of Alternative Needle With Marker Transport Lumen

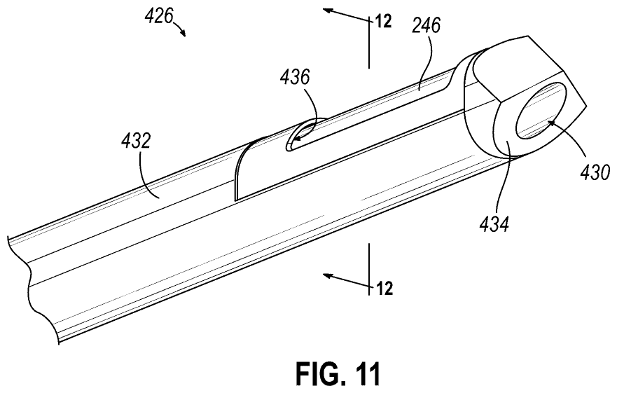

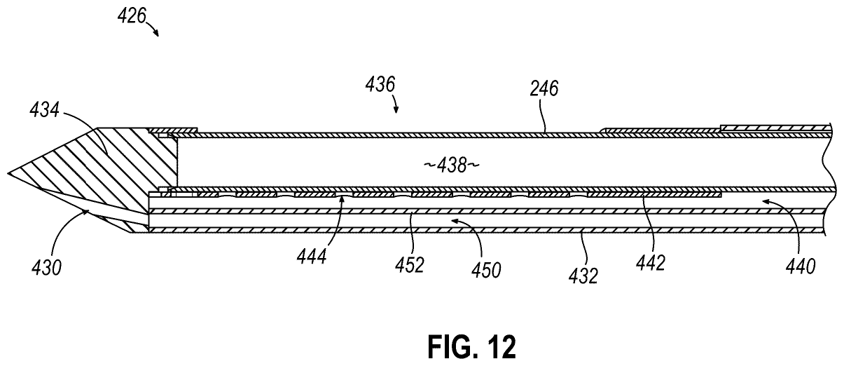

FIGS. 11 and 12 show an example of an alternative needle (426) that may be incorporated into biopsy device (200) described above in lieu of needle (226). Needle (426) of the present version is substantially similar to needle (226) described above, except where otherwise described herein. For instance, like with needle (226) described above, needle (426) of the present version includes an outer cannula (432) having a tissue piercing tip (434) and a transverse tissue receiving aperture (436) (also referred to as a lateral aperture). As with outer cannula (232) described above, the interior of outer cannula (432) of the present version defines a cannula lumen (438) (also referred to as a cutter lumen or axial lumen) and a vacuum lumen (440) (also referred to as a lower lumen or lateral lumen), with a wall (442) separating cannula lumen (438) from vacuum lumen (440). A plurality of fluid openings (444) may be formed in wall (442) to provide fluid communication between cannula lumen (438) and vacuum lumen (440). Of course, as with other components described herein, such openings are merely optional.

Unlike needle (226) described above, needle (426) of the present version includes a dedicated marker transport lumen (450) for deployment of marker (100) rather than through cutter (246). As best seen in FIG. 12, marker transport lumen (450) extends axially the length of needle (426) proximate and parallel to cannula lumen (438) and vacuum lumen (440). Although marker transport lumen (450) is adjacent to vacuum lumen (440) in the present version, it should be understood that in other versions, marker transport lumen (450) may be defined in a variety of positions about outer cannula (432). For instance, marker transport lumen (450) may be oriented to one side in some versions. In such versions, marker transport lumen (450) may be either adjacent to cannula lumen (438), vacuum lumen (440), or both.

Marker transport lumen (450) is defined by an inner wall (452) in the present version. Inner wall (452) may be formed by an integral portion of outer cannula (432) in some versions, or as a separate component in other versions. In the present version, inner wall (452) is configured to fluidly isolate marker transport lumen (450) from other portions of needle (426) such as cannula lumen (428) and vacuum lumen (440). Such fluid isolation may be desirable in some versions to provide a substantially clear linear path for marking without obstruction by biopsy fluid, tissue fragments, and/or etc. In other versions, one or more portions of inner wall (452) may be broken, open, or otherwise in communication with other portions of needle (426). Such a configuration of inner wall (452) may be desirable in some versions to promote ease of assembly or to reduce materials used for needle (426).

Although not shown, it should be understood that the proximal end of marker transport lumen (450) may be configured to communicate either directly with a marker delivery device, tissue sample holder (250), or a port proximate tissue sample holder (250). For instance, in some versions, one or more portions of a marker delivery device may be incorporated into probe (220) or the holster. Such maker delivery device components may be to coupled directly to marker transport lumen (450). In other versions, tissue sample holder (250) may be configured to communicate with marker transport lumen (450) via access port (260) similarly to communication with cutter (246), but offset (e.g., six o'clock position rather than twelve o'clock position). In yet other versions, marker transport lumen (450) may be coupled to a tube (not shown), which may extend proximally from a portion of probe (220). Such a tube may be configured to receive marker (100) directly or portions of a marker delivery device for communication of maker (100) thereto.

The distal end of marker transport lumen (450) is in communication with a marking lumen (430) extending through tissue piercing tip (434). Marking lumen (430) is similar to marking lumen (230) described above in that marking lumen (430) is sized to receive marker (100) so that marker (100) may be deployed through tissue piercing tip (434). Similarly, marking lumen (430) may be positioned relative to tissue piercing tip (434) so as to intersect with only a planar surface and not any cutting edge, as shown in FIG. 11. As described above, such a configuration may be desirable to promote the tissue piercing function of tissue piercing tip (434), while also permitting deployment of marker (100) through tissue piercing tip (434).

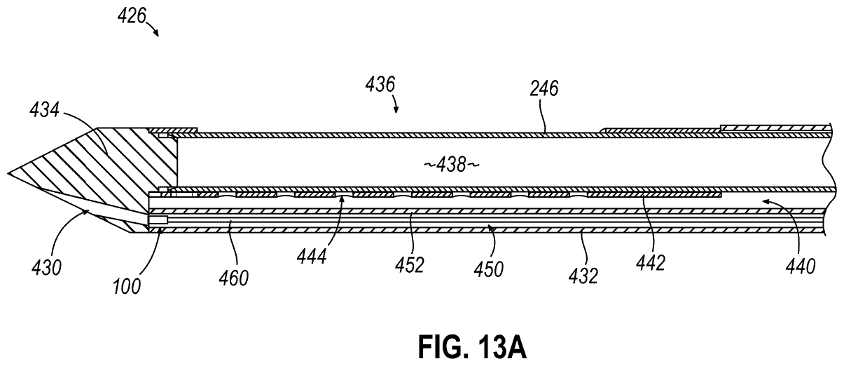

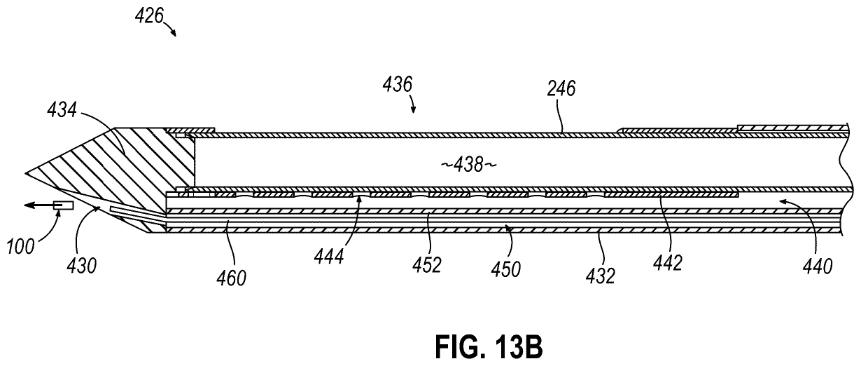

FIGS. 13A through 13B show an example of a use of needle (426) for deployment of marker (100) within tissue. The use with respect to needle (426) of the present version is substantially similar to the use described above with respect to needle (226). For instance, as described above, biopsy device (200) may initially be prepared for marking. Such initial preparations may include, for example, collecting one or more tissue samples, aligning tissue sample holder (250) to communicate with marker transport lumen (450), and/or coupling components of a marker delivery device to biopsy device (200) to communicate marker (100) to biopsy device (200).

Once such initial preparations are completed, marker (100) may be introduced into marker transport lumen (450) and be advanced distally through marker transport lumen (450). In the present use, an elongate push rod (460) is used within marker transport lumen (450) to advance marker (100) distally. It should be understood that push rod (460) may take on a variety of forms. For instance, in some versions, push rod (460) may be flexible to permit deformation of push rod (460) around different components. In other versions, push rod (460) may be generally rigid. Although not shown, push rod (460) may be in communication with various drive components in some versions to facilitate actuation of push rod (460). Such drive components may be incorporated into probe (220) or the holster in some versions or alternatively be separate from biopsy device (200) in other versions.

Although push rod (460) is used to actuate marker (100) in the present use, other suitable mechanisms for actuation may be used. For instance, in some uses, marker (100) may be actuated using a pressurized fluid communicated through marker transport lumen (450). In other uses, marker (100) may be actuated using a pneumatic mechanism. In still other uses, marker (100) may be actuated using various mechanical or electromechanical mechanisms.

Optionally, in some versions, marker (100) and/or push rod (460) may be preloaded as shown in FIG. 13A prior to performing a biopsy procedure. Such a preloaded configuration may be desirable in the present version to simplify the biopsy procedure. Additionally, because marker transport lumen (450) may be fluidly isolated from cannula lumen (438) and/or vacuum lumen (440), the presence of marker (100) and/or push rod (460) may have limited impact on a biopsy procedure, even during sample collection. Although not shown, it should be understood that in preloaded versions, marking lumen (430), marker transport lumen (450), or both may include features configured to maintain the position of marker (100) within needle (426) until deployment of marker (100) is desired. Suitable features configured to maintain the position of maker (100) may include, for example, detents, ridges, protrusions, resilient flaps, and/or etc.

Once marker (100) is advanced through marker transport lumen (440) to marking lumen (430), marker (100) may subsequently be advanced through marking lumen (430) as shown in FIG. 13B. As can be seen, marker (100) may be advanced distally out of tissue piercing tip (434) and into the biopsy site. In the present version, push rod (460) may be used to advance marker (100) through marking lumen (430). In some versions, push rod (460) may be partially extended out of tissue piercing tip (434) to ensure complete deployment of marker (100). Subsequently, push rod (460) may be retracted back into marking lumen (430). After retraction of push rod (460) one or more subsequent markers similar to marker (100) may be optionally deployed using push rod (460).

After completion of marking, needle (426) may be withdrawn from the tissue, while marker (100) may remain in position at the biopsy site. After the biopsy procedure, marker (100) may be used to identify the biopsy site in subsequent follow-up procedures. Identification of marker (100) may be performed using a variety of imaging modalities such as ultrasound, x-ray, MRI, and/or etc.

### IV. Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A system for marking a biopsy site, the system comprising: a biopsy device, the biopsy device including: a body, a needle, the needle extending distally from the body and including a tissue piercing tip, the needle defining an axial lumen and a lateral lumen, the axial lumen being configured to receive a cutter therein, the tissue piercing tip defining a marking lumen in communication with the lateral lumen; and a biopsy site marker, the biopsy site marker including a non-absorbable marker element and a bioabsorbable carrier, the marker element being at least partially disposed within the carrier, the biopsy site marker being configured for receipt within the marking lumen of the needle.

### Example 2

The system of Example 1, the marker lumen extending through a planar surface defined by the tissue piercing tip.

### Example 3

The system of Example 1, the biopsy device further including the cutter, the needle further including an outer cannula, and a lateral aperture defined by a portion of the outer cannula, the cutter being configured to move relative to the lateral aperture to sever one or more tissue samples, the marker lumen being laterally offset relative to a longitudinal axis defined by the cutter.

### Example 4

The system of Example 1, the biopsy device further including the cutter, the needle further including an outer cannula, and a lateral aperture defined by a portion of the outer cannula, the cutter being configured to move relative to the lateral aperture to sever one or more tissue samples, the marker lumen being in communication with a portion of the lateral lumen to define an axial deployment path laterally offset relative to the cutter, the axial deployment path extending from a distal end of the needle to a proximal end of the needle.

### Example 5

The system of Example 1, the biopsy device further including the cutter, the needle further including an outer cannula, an inner wall, and a lateral aperture defined by a portion of the outer cannula, the cutter being configured to move relative to the lateral aperture to sever one or more tissue samples, the inner wall dividing the lateral lumen into a vacuum lumen and a marker transport lumen, the marker lumen being in communication with a portion of the marker transport lumen, the vacuum lumen being in communication with a portion of the cutter.

### Example 6

The system of Example 5, the marker transport lumen being fluidly isolated from the axial lumen and the vacuum lumen.

### Example 7

The system of any of Examples 5 or 6, the marker transport lumen being disposed adjacent to the vacuum lumen.

### Example 8

The system of any of Examples 1 through 7, further comprising a marker actuation element, the marker actuation element being configured to advance the biopsy site marker axially through the needle.

### Example 9

The system of any of Examples 1 through 7, further comprising a marker actuation element, the marker actuation element being configured to advance the biopsy site marker axially through the needle, the marker actuation element including a push rod.

### Example 10

The system of any of Examples 8 or 9, a portion of the marker actuation element being incorporated into the body of the biopsy device.

### Example 11

The system of any of Examples 2 through 10, a portion of the biopsy device defining an access port, the access port being configured to communicate with the marking lumen of the needle.

### Example 12

The system of any of Examples 2 through 10, a portion of the biopsy device defining an access port, the access port being configured to communicate with the marking lumen of the needle, the access port being configured to align with the lateral lumen of the needle to define a continuous path along an axis extending from the access port to the marking lumen.

### Example 13

The system of any of Examples 1 through 10, further comprising a tissue sample holder, the tissue sample holder being in communication with a portion of the needle, the tissue sample holder having an access port configured to permit selective communication with the marking lumen of the needle.

### Example 14

The system of Example 12, the access port of the tissue sample holder being configured to provide a substantially linear path through the tissue sample holder to the needle.

### Example 15

The system of any of Examples 1 through 10, further comprising an access tube, the access tube being in communication with one or more portions of the needle, the access tube being configured to permit communication of the biopsy site marker to the needle.

### Example 16

An apparatus for collecting a biopsy sample and marking tissue, the apparatus comprising: a body; a needle extending distally from the body, the needle including a tissue piercing tip, an outer cannula, and a marker transport lumen defined by a portion of the outer cannula, the marker transport lumen extending along the length of the outer cannula to the tissue piercing tip, the outer cannula defining a lateral aperture proximate the tissue piercing tip; a cutter, the cutter being movable relative to the lateral aperture of the needle to sever one or more tissue samples; a tissue sample holder, the tissue sample holder being in communication with the needle to collect the one or more tissue samples from the needle; and a biopsy site marker, the biopsy site marker including a non-absorbable marker element and a bioabsorbable carrier, the marker element being at least partially disposed within the carrier, the biopsy site marker being pre-loaded in the marker transport lumen prior to a biopsy procedure.

### Example 17

The apparatus of Example 16, the tissue piercing tip defining a marking lumen, the marking lumen extending through a planar surface defined by the tissue piercing tip, the marking lumen being in communication with the marker transport lumen.

### Example 18

The apparatus of Example 16, the tissue piercing tip defining a marking lumen, the marking lumen extending through a planar surface defined by the tissue piercing tip, the marking lumen being in communication with the marker transport lumen, the biopsy site marker being pre-loaded within the marker transport lumen at a position proximate the marking lumen.

### Example 19

The apparatus of any of Examples 16 through 18, the marker transport lumen being laterally offset relative to a longitudinal axis defined by the cutter.

### Example 20

A method for deploying a biopsy site marker in tissue, the method comprising: obtaining a needle of a biopsy device, the needle having the biopsy site marker pre-loaded therein; collecting one or more tissue samples using the needle of the biopsy device to form a biopsy site in the tissue; introducing a drive feature into the needle from a proximal end of the biopsy device to advance the biopsy site marker relative to the needle; and deploying the biopsy site marker through a marker lumen defined by a distal tip of the needle using the drive feature, the act of deploying the biopsy site marker including depositing the biopsy site marker at the biopsy site.

### Example 21

The method of Example 20, adjusting a portion of a tissue sample holder prior to the step of introducing the drive feature to define an axial path extending through the tissue sample holder and needle to the marker lumen.

### Example 22

The method of Example 21, the step of adjusting the tissue sample holder including rotating a rotatable member of the tissue sample holder to move one or more tissue trays relative to the needle.

### Example 23

The method of any of Examples 20 through 22, the step of collecting one or more tissue samples being performed with the biopsy site marker pre-loaded within the needle.

### Example 24

The method of any of Examples 20 through 23, the step of collecting one or more tissue samples including collecting the one or more tissue samples through a lateral aperture defined by the needle.

### Example 25

The method of Example 24, the lateral aperture defined by the needle being opposite of the marker lumen.

### Example 26

The apparatus of any of Examples 16 through 19, the outer cannula further defining a cutter lumen and a vacuum lumen, the cutter lumen being configured to receive the cutter, the vacuum lumen being in communication with a portion of the cutter, the marker transport lumen being positioned adjacent to the vacuum lumen.

### Example 27

The apparatus of any of Examples 16 through 19, the needle further including an inner wall, the outer cannula further defining a cutter lumen and a vacuum lumen, the cutter lumen being configured to receive the cutter, the vacuum lumen being in communication with a portion of the cutter, the marker transport lumen being positioned adjacent to the vacuum lumen, the inner wall being configured to separate the marker transport lumen from the cutter lumen and the vacuum lumen.

### Example 28

The apparatus of any of Examples 16 through 19, the needle further including an inner wall, the outer cannula further defining a cutter lumen and a vacuum lumen, the cutter lumen being configured to receive the cutter, the vacuum lumen being in communication with a portion of the cutter, the marker transport lumen being positioned adjacent to the vacuum lumen, the inner wall being configured to fluidly isolate the marker transport lumen from the cutter lumen and the vacuum lumen.

### Example 29

The apparatus of any of Examples 16 through 19 and 26 thorough 28, further comprising a push rod, the push rod being configured to move within the marker transport lumen to deploy the biopsy site marker into tissue.

### Example 30

The apparatus of Examples 29, the push pod being pre-loaded within the marker transport lumen prior to a biopsy procedure.

### V. Conclusion

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

1. A system for marking a biopsy site, the system comprising:
(a) a biopsy device, the biopsy device including:
(i) a body,
(ii) a needle, the needle extending distally from the body and including a tissue piercing tip, the needle defining an axial lumen and a lateral lumen, the axial lumen being configured to receive a cutter therein, the tissue piercing tip defining a marking lumen in communication with the lateral lumen; and
(b) a biopsy site marker, the biopsy site marker including a non-absorbable marker element and a bioabsorbable carrier, the marker element being at least partially disposed within the carrier, the biopsy site marker being configured for receipt within the marking lumen of the needle.

2. The system of claim 1, the marker lumen extending through a planar surface defined by the tissue piercing tip.

3. The system of claim 1, the biopsy device further including the cutter, the needle further including an outer cannula, and a lateral aperture defined by a portion of the outer cannula, the cutter being configured to move relative to the lateral aperture to sever one or more tissue samples, the marker lumen being laterally offset relative to a longitudinal axis defined by the cutter.

4. The system of claim 1, the biopsy device further including the cutter, the needle further including an outer cannula, and a lateral aperture defined by a portion of the outer cannula, the cutter being configured to move relative to the lateral aperture to sever one or more tissue samples, the marker lumen being in communication with a portion of the lateral lumen to define an axial deployment path laterally offset relative to the cutter, the axial deployment path extending from a distal end of the needle to a proximal end of the needle.

5. The system of claim 1, the biopsy device further including the cutter, the needle further including an outer cannula, an inner wall, and a lateral aperture defined by a portion of the outer cannula, the cutter being configured to move relative to the lateral aperture to sever one or more tissue samples, the inner wall dividing the lateral lumen into a vacuum lumen and a marker transport lumen, the marker lumen being in communication with a portion of the marker transport lumen, the vacuum lumen being in communication with a portion of the cutter.

6. The system of claim 5, the marker transport lumen being fluidly isolated from the axial lumen and the vacuum lumen.

7. The system of claim 5, the marker transport lumen being disposed adjacent to the vacuum lumen.

8. The system of claim 1, further comprising a marker actuation element, the marker actuation element being configured to advance the biopsy site marker axially through the needle.

9. The system of claim 1, further comprising a marker actuation element, the marker actuation element being configured to advance the biopsy site marker axially through the needle, the marker actuation element including a push rod.

10. The system of claim 8, a portion of the marker actuation element being incorporated into the body of the biopsy device.

11. The system of claim 2, a portion of the biopsy device defining an access port, the access port being configured to communicate with the marking lumen of the needle.

12. The system of claim 2, a portion of the biopsy device defining an access port, the access port being configured to communicate with the marking lumen of the needle, the access port being configured to align with the lateral lumen of the needle to define a continuous path along an axis extending from the access port to the marking lumen.

13. The system of claim 1, further comprising a tissue sample holder, the tissue sample holder being in communication with a portion of the needle, the tissue sample holder having an access port configured to permit selective communication with the marking lumen of the needle.

14. The system of claim 12, the access port of the tissue sample holder being configured to provide a substantially linear path through the tissue sample holder to the needle.

15. The system of claim 1, further comprising an access tube, the access tube being in communication with one or more portions of the needle, the access tube being configured to permit communication of the biopsy site marker to the needle.

16. An apparatus for collecting a biopsy sample and marking tissue, the apparatus comprising:
(a) a body;
(b) a needle extending distally from the body, the needle including a tissue piercing tip, an outer cannula, and a marker transport lumen defined by a portion of the outer cannula, the marker transport lumen extending along the length of the outer cannula to the tissue piercing tip, the outer cannula defining a lateral aperture proximate the tissue piercing tip;
(c) a cutter, the cutter being movable relative to the lateral aperture of the needle to sever one or more tissue samples;
(d) a tissue sample holder, the tissue sample holder being in communication with the needle to collect the one or more tissue samples from the needle; and
(e) a biopsy site marker, the biopsy site marker including a non-absorbable marker element and a bioabsorbable carrier, the marker element being at least partially disposed within the carrier, the biopsy site marker being pre-loaded in the marker transport lumen prior to a biopsy procedure.

17. The apparatus of claim 16, the tissue piercing tip defining a marking lumen, the marking lumen extending through a planar surface defined by the tissue piercing tip, the marking lumen being in communication with the marker transport lumen.

18. The apparatus of claim 16, the tissue piercing tip defining a marking lumen, the marking lumen extending through a planar surface defined by the tissue piercing tip, the marking lumen being in communication with the marker transport lumen, the biopsy site marker being pre-loaded within the marker transport lumen at a position proximate the marking lumen.

19. The apparatus of claim 16, the marker transport lumen being laterally offset relative to a longitudinal axis defined by the cutter.

20. A method for deploying a biopsy site marker in tissue, the method comprising:
(a) obtaining a needle of a biopsy device, the needle having the biopsy site marker pre-loaded therein;
(b) collecting one or more tissue samples using the needle of the biopsy device to form a biopsy site in the tissue;
(c) introducing a drive feature into the needle from a proximal end of the biopsy device to advance the biopsy site marker relative to the needle; and
(d) deploying the biopsy site marker through a marker lumen defined by a distal tip of the needle using the drive feature, the act of deploying the biopsy site marker including depositing the biopsy site marker at the biopsy site.

21.-30. (canceled)
